(19) United States
(12) Patent Application Publication
(10) Pub. No.: US 2022/0051752 A1
(43) Pub. Date: Feb. 17, 2022

(54) PREDICTING IMMUNOGENIC PEPTIDES USING STRUCTURAL AND PHYSICAL MODELING

(71) Applicant: University of Notre Dame du Lac, South Bend, IN (US)

(72) Inventors: Brian Baker, South Bend, IN (US); Tim Riley, South Bend, IN (US)

(21) Appl. No.: 17/312,134

(22) PCT Filed: Dec. 6, 2019
(86) PCT No.: PCT/US2019/064959

§ 371 Date: Jun. 9, 2021

Related U.S. Application Data
(60) Provisional application No. 62/777,638, filed on Dec. 10, 2018.

(51) Int. Cl. G16B 15/00; G16B 40/20

(52) U.S. Cl. CPC G16B 15/00; G16B 40/20

(57) ABSTRACT

Disclosed herein are methods for predicting immunogenicity of a candidate peptide. The method comprises obtaining a three-dimensional candidate structural representation of the candidate peptide bound to an antigen presenting molecule; obtaining a plurality of candidate measurements; and predicting, with an electronic processor, the immunogenicity of the candidate peptide based upon the plurality of candidate measurements. Further disclosed herein are methods for producing vaccines. The method for producing a vaccine comprises predicting immunogenicity of one or more candidate peptides using the methods described herein, and producing a vaccine comprising one or more peptides predicted to be immunogenic.

### CROSS-REFERENCE TO RELATED APPLICATIONS

This claims priority to U.S. Provisional Patent Application No. 62/777,638, filed on Dec. 10, 2018, the entire contents of which are fully incorporated herein by reference.

### STATEMENT OF GOVERNMENT INTEREST

This invention was made with government support under grant R35GM118166 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 12,450 bytes ASCII (Text) file named “18-072-092012-9093-WO01-SEQ-LIST ST25.txt,” created on Dec. 4, 2019.

### TECHNICAL FIELD

The present disclosure relates to methods for predicting immunogenic peptides using structural and physical modeling. In particular, the methods disclosed herein may be used to predict immunogenic cancer neoantigens.

### BACKGROUND

Successful therapeutic vaccination relying on peptide antigens presented to T cells of the immune system is a longstanding goal for cancer immunotherapy. DNA sequencing and advances in immunoinformatics have led to the identification of neoantigens incorporating nonsynonymous mutations that differentiate tumors from healthy tissues. Following sequencing of tumor material, potential neoantigens have been identified via bioinformatic approaches that predict processing and presentation by MHC proteins, and more recently, mass spectrometry. However, it is becoming increasingly recognized that, even after taking tolerance mechanisms into account, not all well-presented peptides are strongly immunogenic, indicating the existence of peptide features that influence T cell recognition independently of MHC binding. Accordingly, effective means for identifying peptides that are immunogenic and can thus promote tumor rejection are needed.

### SUMMARY

Disclosed herein are methods for predicting immunogenicity of a candidate peptide. The method comprises obtaining a three-dimensional candidate structural representation of the candidate peptide bound to an antigen presenting molecule; obtaining a plurality of candidate measurements, wherein each candidate measurement is associated with at least one feature of the candidate structural representation; and predicting, with an electronic processor, the immunogenicity of the candidate peptide, wherein the electronic processor is configured to predict the immunogenicity of the candidate peptide based upon the plurality of candidate measurements. Further disclosed herein are methods for producing vaccines. The method for producing a vaccine comprises predicting immunogenicity of one or more candidate peptides using the methods described herein, and producing a vaccine comprising one or more peptides predicted to be immunogenic.

Other aspects of the disclosure will become apparent by consideration of the detailed description and accompanying drawings.

### BRIEF DESCRIPTIONS OF THE DRAWINGS

This patent or application file contains at least one drawing executed in color. Copies of this patent or patent application publication with color drawing(s) will be provided by the Office upon request and payment of the necessary fee.

FIGS. 1a-c show rapid structural modeling for peptide/HLA-A2 complexes. FIG. 1a is a graph showing modeling performance for 62 structures, showing RMSD for modeled vs. crystallized peptides in a box and whisker plot. The left shows RMSD calculations for a carbons only; the right shows all peptide atoms. Boxes illustrate the 1^{st} and 3^{rd} quartiles, with a horizontal line at the median and a red star at the mean. Whiskers show 1.5 of the interquartile range. FIG. 1b shows structural images of representative models and their corresponding structures. The top shows the model of NLVPAVATV (SEQ ID NO: 1), which superimposes on the crystal structure with a full atom RMSD of 1.08 Å. The bottom shows the model of LAGIGILTV (SEQ ID NO: 2), which superimposes on the structure with a full atom RMSD of 2.59 Å. For the latter case, the leucine at position 1 forces the nonameric peptide to bind in a register-shifted decameric configuration, with the p1 leucine in the B-pocket. The modeling procedure did not permit such drastic sampling. FIG. 1c is a graph showing correlation between exposed peptide hydrophobic surface area in the models vs. the crystallographic structures. The two sets of data correlate with an R value of 0.63.

FIGS. 2a-2b show characteristics of peptides in the training set. FIG. 2a shows sequence logos of immunogenic peptides (top), HeLa self-peptides (middle), and HLA-A2 non-binding peptides (bottom). FIG. 2b is a graph showing comparison of the hydrophobicity of each peptide position in the immunogenic and self-peptide datasets (presented as immunogenic—self) as determined using the Wimley-White hydropathy index. Values less than zero (below dashed line) indicate greater hydrophobicity in the immunogenic dataset. p values are indicated where the differences are statistically significant.

FIGS. 3a-c show the process and architecture of the structure-based immunogenicity neural network. FIG. 3a shows the process begins with a peptide sequence, which is used to generate a model of the peptide/HLA-A2 three-dimensional structure using Rosetta. FIG. 3b shows analysis of the modeled structure yields energetic and topographical information, which are used as inputs for the structure-based immunogenicity neural network (SBIN). FIG. 3c shows SBIN architecture, with 81 structure-derived inputs shown on the left (seven for each peptide position, 18 for the overall complex). A single hidden layer is present with five hidden neurons, along with two constant bias nodes. Black lines give positive weights, grey lines negative weights, with line width indicating weight magnitude.

FIGS. 4a-b show performance of the structure-based immunogenicity neural network in categorizing peptide immunogenicity. FIG. 4a is a graph showing performance of SBIN compared to other approaches in evaluating the training data as demonstrated by a receiver operating characteristic curve. The area under the curve (AUC) for each approach gives the probability that the approach will more favorably score an immunogenic peptide than a non-immunogenic peptide. In evaluating the training data of 3955 peptides SBIN outperformed a sparse matrix network based on sequence alone, the IEDB prediction tool, NetTepi, and netMHCpan 4.0 in both affinity and ligand likelihood modes. FIG. 4b is a graph showing that against a neoantigen dataset of 291 nonameric peptides SBIN performed less favorably, but still outperformed the other approaches.

FIGS. 5a-d show modeled structures of select neoantigens and their wild-type counterparts. FIG. 5a shows the neoantigen LIIPFIHLI (SEQ ID NO: 3) substitutes a phenylalanine for a cysteine at position 5. The position 5 side chain is predicted to extend from the top of a bulge in the peptide, and the mutation results in an increase in exposed hydrophobic surface of 90 Å². FIG. 5b shows the neoantigen AVGSYVYSV (SEQ ID NO: 4) substitutes a tyrosine for a histidine at position 5. The position 5 side chain is again predicted to extend from the top of the bulge in the peptide. The mutation results in the exposure of only 11 Å² of additional hydrophobic surface but also eliminates a positive charge in the central portion of the peptide while providing hydrogen bonding opportunities for an incoming TCR. FIG. 5c shows the neoantigen ILNAMIAKI (SEQ ID NO: 5) substitutes an alanine for a threonine at position 7. The position 7 side chain is predicted to lie in the interface between the peptide backbone and the HLA-A2 α2 helix. The mutation reduces exposed hydrophobic surface area by 7 Å² but also “unmasks” the position 7 amide nitrogen as indicated by the arrow, providing new hydrogen bonding opportunities for an incoming TCR. FIG. 5d shows the neoantigen KLSHQLVLL (SEQ ID NO: 6) substitutes a leucine for a proline at position 6 of the peptide. The position 6 side chain in the neoantigen is predicted to extend towards the base of the HLA-A2 peptide binding groove, whereas the proline in the wild-type peptide is predicted to lie in the interface between the backbone and the HLA-A2 α1 helix. The mutation again predicts a reduction in exposed hydrophobic surface area (16 Å²), as well as the exposure of a new hydrogen bonding site.

FIG. 6 is a graph showing percent solvent exposed surface area at amino acid positions 1-9 following TCR binding to nonameric peptide/HLA-A2 complexes.

### DETAILED DESCRIPTION

Disclosed herein are methods for predicting immunogenicity of a candidate peptide. For example, disclosed herein are methods for predicting immunogenicity of a cancer neoantigen.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Immunogenicity” as used herein refers to the ability of a substance to invoke an immune response. The immune response may be in the body, a model organism such as a mouse, or in vitro such as in cultured immune cells. As used herein, “immunogenic” refers to peptides that invokes responses from immune cells. As used herein, “non-immunogenic” refers to peptides that do not invoke responses from immune cells.

### 2. METHODS FOR PREDICTING IMMUNOGENICITY

Disclosed herein are methods for predicting immunogenicity of one or more candidate peptides. The methods described herein explicitly contemplate predicting the immunogenicity of one candidate peptide or predicting the immunogenicity of multiple candidate peptides. The methods comprise obtaining a three-dimensional candidate structural representation of the candidate peptide bound to an antigen presenting molecule. The three-dimensional candidate structural representation may be generated. For example, the three-dimensional candidate structural representation may be generated using any suitable software known in the art. Alternatively, the three-dimensional candidate structural representation may be obtained from any suitable source, such as a database.

The method further comprises obtaining a plurality of candidate measurements. Each candidate measurement is associated with at least one feature of the candidate structural representation. For example, the method may comprise obtaining a plurality of candidate measurements selected from the group consisting of solvent accessible surface areas, solvation energies, hydrophobicity, electrostatic interactions, and van der Waals interactions. These measurements are listed as examples only and are not intended in any way to be limiting. Other suitable measurements may be used in addition or alternatively to these example measurements. For example, other suitable measurements are provided in Table 1.

The method further comprises predicting, with an electronic processor, the immunogenicity of the candidate peptide. The electronic processor may be a microprocessor, an application-specific integrated circuit (ASIC), or other suitable electronic device. The electronic processor executes computer-readable instructions (“software”). The software may include firmware, one or more applications, program data, filters, rules, one or more program modules, and other executable instructions. For example, the software may include instructions and associated data for performing a set of functions including the methods described herein. The electronic processor may be configured to predict the immunogenicity of the candidate peptide based upon the plurality of candidate measurements.

The electronic processor may be further configured to predict the immunogenicity of the candidate peptide based upon a plurality of reference measurements. Each reference measurement may be associated with at least one feature of one or more reference structural representations. Each reference structural representation is a three-dimensional representation of a reference peptide bound to the antigen presenting molecule. Each reference peptide may be a known immunogenic peptide or a known non-immunogenic peptide. Each reference measurement may be selected from the group consisting of solvent accessible surface areas, solvation energies, hydrophobicity, electrostatic interactions, and van der Waals interactions. These measurements are listed as examples only and are not intended in any way to be limiting. Other suitable measurements may be used in addition or alternatively to these example measurements. For example, other suitable measurements are provided in Table 1. In some embodiments, the electronic processor is further configured to predict the immunogenicity of the candidate peptide based upon whether each reference peptide is an immunogenic peptide or a non-immunogenic peptide.

The electronic processor may be configured to predict the immunogenicity of the candidate peptide using a machine-learned model trained to predict immunogenicity of the candidate peptide using the plurality of reference measurements. Machine learning generally refers to the ability of a computer program to learn without being explicitly programmed. In some embodiments, a computer program is configured to construct a model (one or more algorithms) based on example inputs. Machine learning involves presenting a computer program with example inputs and their desired (for example, actual) outputs. The computer program is configured to learn a general rule (a model) that maps the inputs to the outputs. The computer program may be configured to perform machine learning using various types of methods and mechanisms. For example, the computer program may perform machine learning using decision tree learning, association rule learning, artificial neural networks, inductive logic programming, support vector machines, clustering, Bayesian networks, reinforcement learning, representation learning, similarity and metric learning, sparse dictionary learning, or genetic algorithms.

The antigen presenting molecule may be any desired antigen presenting molecule. For example, the antigen presenting molecule may be an MEW molecule. In some embodiments, the antigen presenting molecule is a class I MHC molecule or a class II MEW molecule. For example, the antigen presenting molecule may be HLA-A2.

The candidate peptide may be any desired candidate peptide. For example, the candidate peptide may be a neoantigen, a viral peptide, a non-mutated self peptide, or a post-translationally modified peptide.

### 3. METHODS FOR PRODUCING VACCINES

Further disclosed herein are methods for producing vaccines. The methods for producing vaccines comprise predicting immunogenicity of one or more candidate peptides using the methods described herein, and producing a vaccine comprising one or more candidate peptides predicted to be immunogenic by the method. The methods described herein may be used to produce a vaccine for any desired disease or condition. For example, the methods described herein may be used to produce a cancer vaccine. In accordance with such embodiments, the method may be used to predict immunogenicity of one or more neoantigens, and the neoantigens predicted to be immunogenic may be used in the subsequent production of a cancer vaccine.

### 4. EXAMPLES

The following Examples are offered as illustrative as a partial scope and particular embodiments of the disclosure and are not meant to be limiting of the scope of the disclosure.

### Example 1

### Methods

Structural modeling of HLA A2 presented peptides: Structural modeling of peptide/HLA-A2 complexes was performed with PyRosetta using the Talaris2014 energy function. The desired peptide sequence was computationally introduced into HLA-A2, using PDB ID 3QFD (2^{nd} molecule in the asymmetric unit) as a template for nonamers and 1JF1 as a template for decamers. This was followed by 50 Monte Carlo-based simulated annealing sidechain and peptide backbone minimization steps using the LoopMover_Refine_CCD protocol, generating 20 independent decoys per peptide. The large number of resulting packing operations introduced some minor variability when scoring the models. Therefore, the unweighted score terms for the three lowest scoring trajectories were averaged and used for neural network inputs.

Collection of data sets: The structural database for evaluating modeling strategies consisted of high resolution (<3.0 Å) nonameric or decameric peptide/HLA-A2 structures within the PDB. Structures in this dataset were selected for strong electron density as determined by visual inspection using COOT for calculating 2Fₒ-F_{c} density maps. The final database contained 62 structures presenting different peptide epitopes (56 nonamers and 6 decamers). For structures with multiple molecules in the asymmetric unit, RMSDs of modeled peptides were calculated to all molecules and the lowest RMSD value was reported.

The neural network training set contained 3955 nonameric peptides collected from published sources. For self-peptides categorized as non-immunogenic, lists of peptides identified via mass spectrometry analysis of human HeLa cells transfected with soluble HLA-A2 were used. HLA-A2 incompatible peptides (IC₅₀>50,000 nM) were downloaded from IEDB. Immunogenic peptides were stringently selected from IEDB to ensure quality of data and minimize false positives by restricting selected peptides to those with a positive IFN-γ ELISpot with a response frequency starting at 50%. The test dataset was derived from a review of validated neoantigens. Only nonameric peptides presented by HLA-A2 were selected for evaluation, resulting in a dataset consisting of 291 candidate neoantigens.

Artificial neural network training: Two-layer feed-forward networks were trained with the scaled conjugate gradient back-propagation training tool in Matlab 2017b. Training and evaluation of neural network architectures was performed using a nested five-fold cross-validation procedure. The peptides in the training dataset were split into five sets of training, validation, and test data. The splitting was performed such that all sets have approximately the same distribution of non-binding, self, and immunogenic peptides. With the binary classification criteria of immunogenic or non-immunogenic (with non-immunogenic incorporating self and non-binding peptides), the training data were used to perform feed-forward and back propagation. The validation set defined the stopping criteria for the network training, and the test set evaluated performance via AUC. Sets were rotated to ensure each was used in training, validation, and testing. The average AUC of all the test sets, reported as an indicator of overall performance, was 0.69. To maintain an equal distribution of classifiers and eliminate bias for non-immunogenic peptides, immunogenic peptides in the training sets, but not testing or validation sets, were randomly oversampled.

The neural network architecture used was a conventional feed-forward network with an input layer containing 80-117 neurons, one hidden layer with 1-10 neurons, and a single neuron output layer. The neurons in the input layer describe structural and structure-derived energetic-features of the 9 amino acids in the peptide sequence, with each amino acid represented by up to 11 neurons. The remaining 18 neurons describe global structural and structure-derived energetic features of the entire peptide/HLA-A2 complex. The structural and energetic features were those that comprise the Talaris2014 energy function or derived from the structure as listed in Table 1. For each of the five training and test sets, a series of network trainings were performed each with a different number of hidden neurons (2, 3, 4, 6, 8, and 10) and a different number of input neurons. Finally, a single network with the highest test performance was finally selected.

For developing a control network that considered peptide sequence only, peptide sequences were encoded in 20×9 sparse matrices. These matrices were used to train a network of the same architecture (except that it relied on 180 input nodes) that was subject to the same cross validation procedure.

TABLE 1
Structural and structure-derived terms used for training the structure
based immunogenicity network. Energetic terms are those
that comprise the Talaris2014 energy function

| Global energetic terms | |
|---|---|
| describing entire | |
| peptide/MHC complex | Description |
| total_score | Total Talaris 2014 total energy |
| fa_atr | Total Lennard-Jones attractive |
| fa_rep | Total Lennard-Jones repulsive |
| fa_sol | Total Lazaridus-Karplus solvation energy |
| fa_intra_rep | Total Lennard-Jones repulsive between |
| | atoms of same residue |
| fa_elec | Total Coulombic electrostatic potential |
| pro_close | Total proline ring closure energy |
| hbond_sr_bb | Total backbone-backbone hydrogen bond |
| | energy, close in structure |
| hbond_lr_bb | Total backbone-backbone hydrogen bond |
| | energy, distant in structure |
| hbond_bb_sc | Total sidechain-backbone hydrogen bond |
| | energy |
| hbond_sc | Total sidechain-sidechain hydrogen bond |
| | energy |
| dslf_fal3 | Total disulfide geometry potential |
| rama | Total ramachandran preference energy |
| omega | Total omega dihedral energy in the |
| | backbone |
| fa_dun | Total internal energy of sidechain rotamers |
| | as derived from Dunbrack's statistics |
| p_aa_pp | Total probability of amino acid at phipsi |
| yhh_planarity | Total torsional potential for Tyrosine |
| ref | Total of reference energies for each amino |
| | acid |
| Energetic terms at | |
| the level of each | |
| peptide amino acid | Description |
| fa_atr | Lennard-Jones attractive (between position |
| | atoms and every other atom of pMHC) |
| fa_rep | Lennard-Jones repulsive (between position |
| | atoms and every other atom of pMHC) |
| fa_sol | Lazaridus-Karplus solvation energy for |
| | position |
| fa_intra_ rep (excluded | Lennard-Jones repulsive between atoms of |
| after cross-validation) | same residue |
| fa_elec | Coulombic electrostatic potential (between |
| | position and every other atom of pMHC) |
| rama (excluded after | Ramachandran preferences |
| cross-validation) | |
| fa_dun (excluded after | Internal energy of sidechain rotamers as |
| cross-validation) | derived from Dunbrack's statistics |
| p_aa_pp (excluded after | Probability of amino acid at phipsi |
| cross-validation) | |
| ref | Amino acid reference energy for position |
| Additional amino acid | |
| level terms (structure | |
| derived, non-energetic) | Description |
| sasa | Solvent accessible surface area |
| hsasa | Hydrophobic solvent accessible |
| | surface area |

### Example 2

### Development and Performance of a Rapid pMHC Modeling Strategy

To develop a rapid structural modeling strategy, an extensive list of peptide/WIC structures within the PDB were compiled. Analysis was restricted to high resolution HLA-A2 structures with good electron density throughout the length of the peptide. To emphasize structural differences emerging from amino acid changes, the database was further narrowed by pairing each peptide/HLA-A2 complex with at least one other in which the peptide differed by only a single amino acid, either as a substitution or transposition. The final database contained 62 structures presenting distinct peptide epitopes (56 nonamers and 6 decamers) (Table 2).

TABLE 2
Structures utilized in benchmarking
structural modeling

| | | Talaris | FA | Ca |
|---|---|---|---|---|
| PDB | | 2014 | RMSD | RMSD |
| Entry | Sequence | energy^{a} | (Å)^{b} | (Å)^{c} |
| 3qfd | AAGIGILTV | −488.09 | 0.69 | 0.45 |
| | (SEQ ID NO: 7) | | | |
| 1jht | ALGIGILTV | −488.69 | 0.76 | 0.35 |
| | (SEQ ID NO: 8) | | | |
| 1b0g | ALWGFFPVL | −446.86 | 3.11 | 0.96 |
| | (SEQ ID NO: 9) | | | |
| 1i7u | ALWGFVPVL | −460.77 | 3.39 | 1.86 |
| | (SEQ ID NO: 10) | | | |
| 1i7t | ALWGVFPVL | −464.75 | 2.91 | 0.98 |
| | (SEQ ID NO: 11) | | | |
| 3mrj | CINGMCWTV | −476.58 | 2.00 | 0.66 |
| | (SEQ ID NO: 12) | | | |
| 3mrg | CINGVCWTV | −471.70 | 1.68 | 0.89 |
| | (SEQ ID NO: 13) | | | |
| 3mr1 | CINGVVWTV | −473.66 | 1.31 | 0.83 |
| | (SEQ ID NO: 14) | | | |
| 3mrh | CISGVCWTV | −477.16 | 1.67 | 0.78 |
| | (SEQ ID NO: 15) | | | |
| 2gt9 | EAAGIGILTV | −507.03 | 0.72 | 0.20 |
| | (SEQ ID NO: 16) | | | |
| 2gtw | LAGIGILTV | −484.17 | 2.59 | 2.23 |
| | (SEQ ID NO: 2) | | | |
| 1jf1 | ELAGIGILTV | −508.13 | 0.65 | 0.13 |
| | (SEQ ID NO: 17) | | | |
| 4jfo | ALAGIGILTV | −512.17 | 0.60 | 0.29 |
| | (SEQ ID NO: 18) | | | |
| 3mro | ELAGWGILTV | −505.14 | 0.92 | 0.29 |
| | (SEQ ID NO: 19) | | | |
| 5hhp | GILEFVFTL | −467.52 | 1.45 | 0.31 |
| | (SEQ ID NO: 20) | | | |
| 2v11 | GILGFVFTL | −467.42 | 2.35 | 0.95 |
| | (SEQ ID NO: 21) | | | |
| 5hhn | GILGLVFTL | −468.41 | 1.64 | 0.72 |
| | (SEQ ID NO: 22) | | | |
| 5hhq | GIWGFVFTL | −436.19 | 1.21 | 0.52 |
| | (SEQ ID NO: 23) | | | |
| 3mrf | GLCPLVAML | −470.61 | 1.50 | 0.68 |
| | (SEQ ID NO: 24) | | | |
| 3mre | GLCTLVAML | −470.29 | 1.77 | 1.05 |
| | (SEQ ID NO: 25) | | | |
| 2x4u | ILKEPVHGV | −280.78 | 0.84 | 0.35 |
| | (SEQ ID NO: 26) | | | |
| 1i1f | FLKEPVHGV | −289.95 | 0.84 | 0.23 |
| | (SEQ ID NO: 27) | | | |
| 1i1y | YLKEPVHGV | −295.73 | 1.00 | 0.39 |
| | (SEQ ID NO: 28) | | | |
| 1eez | ILSALVGIL | −472.34 | 1.85 | 1.04 |
| | (SEQ ID NO: 29) | | | |
| 1eey | ILSALVGIV | −470.09 | 1.25 | 0.68 |
| | (SEQ ID NO: 30) | | | |
| 1tvh | IMDQVPFSV | −478.66 | 1.67 | 0.80 |
| | (SEQ ID NO: 31) | | | |
| 1tvb | ITDQVPFSV | −480.64 | 1.55 | 0.77 |
| | (SEQ ID NO: 32) | | | |
| 3v5h | KVAEIVHFL | −469.46 | 1.79 | 0.84 |
| | (SEQ ID NO: 33) | | | |
| 3v5d | KVAELVHFL | −434.03 | 1.59 | 0.36 |
| | (SEQ ID NO: 34) | | | |
| 3v5k | KVAELVWFL | −473.24 | 2.53 | 0.88 |
| | (SEQ ID NO: 35) | | | |
| 3pw1 | LGYGFVNYI | −435.88 | 3.06 | 1.29 |
| | (SEQ ID NO: 36) | | | |
| 3pwn | LLYGFVNYI | −431.36 | 2.70 | 0.91 |
| | (SEQ ID NO: 37) | | | |
| 3pwj | LLYGFVNYV | −464.42 | 2.60 | 1.43 |
| | (SEQ ID NO: 38) | | | |
| 2git | LLFGKPVYV | −433.12 | 2.34 | 0.90 |
| | (SEQ ID NO: 39) | | | |
| 1im3 | LLFGYPVYV | −383.66 | 2.44 | 0.69 |
| | (SEQ ID NO: 40) | | | |
| 3mrc | NLVPMCATV | −476.86 | 2.67 | 1.47 |
| | (SEQ ID NO: 41) | | | |
| 3mrd | NLVPMGATV | −472.02 | 1.20 | 0.93 |
| | (SEQ ID NO: 42) | | | |
| 3gsw | NLVPMVAAV | −469.65 | 1.52 | 0.89 |
| | (SEQ ID NO: 43) | | | |
| 3gso | NLVPMVATV | −474.97 | 1.14 | 0.38 |
| | (SEQ ID NO: 44) | | | |
| 3gsx | NLVPMVAVV | −473.99 | 1.07 | 0.51 |
| | (SEQ ID NO: 45) | | | |
| 3mrb | NLVPMVHTV | −477.22 | 1.23 | 0.65 |
| | (SEQ ID NO: 46) | | | |
| 3gsv | NLVPQVATV | −475.90 | 1.39 | 0.75 |
| | (SEQ ID NO: 47) | | | |
| 3gsq | NLVPSVATV | −478.06 | 1.16 | 0.65 |
| | (SEQ ID NO: 48) | | | |
| 3gsu | NLVPTVATV | −474.65 | 1.21 | 0.74 |
| | (SEQ ID NO: 49) | | | |
| 3gsr | NLVPVVATV | −465.23 | 1.07 | 0.61 |
| | (SEQ ID NO: 50) | | | |
| 3mr9 | NLVPAVATV | −476.83 | 1.08 | 0.70 |
| | (SEQ ID NO: 1) | | | |
| 3mgo | RLYQNPTTYI | −276.50 | 1.96 | 0.66 |
| | (SEQ ID NO: 51) | | | |
| 3mgt | KLYQNPTTYI | −269.34 | 2.78 | 1.64 |
| | (SEQ ID NO: 52) | | | |
| 1s8d | SLANTVATL | −475.50 | 1.33 | 0.71 |
| | (SEQ ID NO: 53) | | | |
| 2v2x | SLFNTVATL | −475.95 | 2.60 | 1.49 |
| | (SEQ ID NO: 54) | | | |
| 1s9x | SLLMWITQA | −470.59 | 2.08 | 1.10 |
| | (SEQ ID NO: 55) | | | |
| 1s9w | SLLMWITQC | −470.56 | 2.34 | 1.27 |
| | (SEQ ID NO: 56) | | | |
| 3k1a | SLLMWITQL | −459.05 | 1.87 | 0.99 |
| | (SEQ ID NO: 57) | | | |
| 1s9y | SLLMWITQS | −469.11 | 2.07 | 1.08 |
| | (SEQ ID NO: 58) | | | |
| 1t1x | SLYLTVATL | −464.44 | 2.00 | 0.60 |
| | (SEQ ID NO: 59) | | | |
| 1t20 | SLYNTIATL | −469.84 | 2.11 | 0.71 |
| | (SEQ ID NO: 60) | | | |
| 2v2w | SLYNTVATL | −461.86 | 2.04 | 0.63 |
| | (SEQ ID NO: 61) | | | |
| 1t1y | SLYNVVATL | −365.42 | 2.05 | 0.74 |
| | (SEQ ID NO: 62) | | | |
| 3ft3 | VLHDDLLEA | −457.06 | 1.87 | 0.74 |
| | (SEQ ID NO: 63) | | | |
| 3ft4 | VLRDDLLEA | −442.43 | 2.23 | 1.02 |
| | (SEQ ID NO: 64) | | | |
| 3myj | YMFPNAPYL | −394.92 | 2.23 | 0.81 |
| | (SEQ ID NO: 65) | | | |
| 3hpj | RMFPNAPYL | −463.57 | 1.85 | 1.00 |
| | (SEQ ID NO: 66) | | | |

^{a}Total energy of modeled peptide/HLA-A2 complex as scored by the Talaris 2014 score function in Rosetta energy units
^{b}full atom RMSD of modeled peptide to structure
^{c}a carbon RMSD of modeled peptide to structure

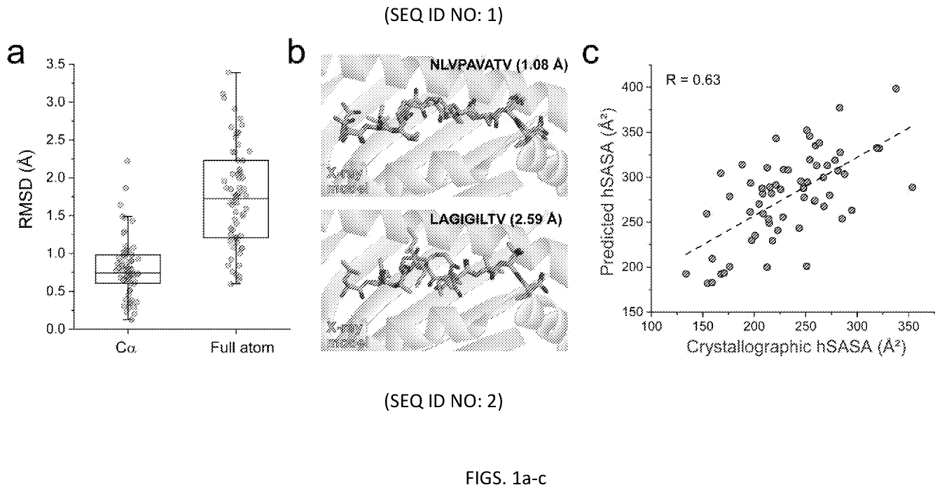

Modeling speed was prioritized over complexity. Nonameric and decameric peptides bound to class I MHC proteins adopt relatively conserved backbone conformations. Therefore, each complex in the database was modeled by threading the desired peptide sequence into template HLA-A2 structures, followed by Monte-Carlo-based conformational sampling and energy minimization for side chains and the peptide backbones utilizing Rosetta. This approach, which required approximately 10 minutes per model on 2016-vintage CPU hardware, predicted the experimentally determined structures with a mean peptide Cα root mean square deviation (RMSD) of 0.8 Å and full-atom RMSD of 1.8 Å (FIG. 1A; Table 2). The greatest discrepancy between modeled and actual structures was an unusual register-shifted nonameric peptide (LAGIGILTV) (SEQ ID NO: 2) which, compared to the native peptide (AAGIGILTV) (SEQ ID NO: 7), left the p1 pocket of the HLA-A2 molecule empty in the crystal structure, so the nonameric peptide adopted a decameric configuration (FIG. 1B). The rapid modeling procedure was not able to sample such dramatic conformational shifts, and thus the model of this peptide resembled more traditional nonameric peptide/WIC structures.

Other approaches to model peptides in class I WIC binding grooves have incorporated docking, molecular dynamics simulations, protein threading, or combinations of these methods. These other methods have reported Cα or full-atom RMSD values between model and experiment within the approximate range of 1-2 Å. This approach thus compares favorably with or even outperforms other efforts.

Given recent attention on the role of exposed surface features in the immunogenicity of MHC-presented peptides, it was evaluated how the modeling procedure recovered peptide hydrophobic solvent accessible surface area (hSASA). After comparing models and structures, the correlation between predicted and experimental hSASA was 0.63 (FIG. 1C). The modeling procedure provides a good approximation of peptide structural properties within the binding groove of HLA-A2 and the changes that occur upon mutation.

### Example 3

### Collecting a Broad Peptide Dataset

To test whether consideration of structural features could lead to improved immunogenicity predictions, a large peptide database that contains immunogenic and non-immunogenic peptides was developed. While the IEDB has records for immunogenic peptides, it contains limited data on peptides that are poorly immunogenic yet still well-presented by WIC proteins. To account for such peptides, lists of peptides identified via proteomic analyses of human HeLa cells transfected with soluble HLA-A2 were used. For modeling accuracy, the dataset focused exclusively on nonamers, yielding a dataset of 2756 HLA-A2-presented self-peptides. While this dataset will necessarily include peptides that would be efficiently recognized by TCRs and thus drive negative selection, it was hypothesized it would also be enriched in peptides that are positively selected yet still do not possess the structural or chemical features to promote efficient TCR recognition. To the set of self-peptides, 155 immunogenic peptides listed in the IEDB were added, selected by filtering for HLA-A2-presented human nonamers with an IFN-□ ELISPOT response frequency of 50 or higher. The immunogenic peptide dataset primarily included epitopes from viral sources, although humans and other organisms were also represented (Table 3).

TABLE 3
Structural and structure-derived terms used for training the
structure based immunogenicity network. Energetic terms are
those that comprise the Talaris2014 energy function

| Global energetic terms | |
|---|---|
| describing entire | |
| peptide/MHC complex | Description |
| total_score | Total Talaris 2014 total energy |
| fa_atr | Total Lennard-Jones attractive |
| fa_rep | Total Lennard-Jones repulsive |
| fa_sol | Total Lazaridus-Karplus solvation energy |
| fa_intra_rep | Total Lennard-Jones repulsive between |
| | atoms of same residue |
| fa_elec | Total Coulombic electrostatic potential |
| pro_close | Total proline ring closure energy |
| hbond_sr_bb | Total backbone-backbone hydrogen bond |
| | energy, close in structure |
| hbond_lr_bb | Total backbone-backbone hydrogen bond |
| | energy, distant in structure |
| hbond_bb_sc | Total sidechain-backbone hydrogen bond |
| | energy |
| hbond_sc | Total sidechain-sidechain hydrogen bond |
| | energy |
| dslf_fa_13 | Total disulfide geometry potential |
| rama | Total ramachandran preference energy |
| omega | Total omega dihedral energy in the |
| | backbone |
| fa_dun | Total internal energy of sidechain rotamers |
| | as derived from Dunbrack's statistics |
| p_aa_pp | Total probability of amino acid at phipsi |
| yhh_planarity | Total torsional potential for Tyrosine |
| ref | Total of reference energies for each amino |
| | acid |
| Energetic terms at | |
| the level of each | |
| peptide amino acid | Description |
| fa_atr | Lennard-Jones attractive (between position |
| | atoms and every other atom of pMHC) |
| fa_rep | Lennard-Jones repulsive (between position |
| | atoms and every other atom of pMHC) |
| fa_sol | Lazaridus-Karplus solvation energy for |
| | position |
| fa_intra_rep (excluded | Lennard-Jones repulsive between atoms of |
| after cross-validation) | same residue |
| fa_elec | Coulombic electrostatic potential (between |
| | position and every other atom of pMHC) |
| Rama (excluded after | Ramachandran preferences |
| cross-validation) | |
| fa_dun (excluded after | Internal energy of sidechain rotamers as |
| cross-validation) | derived from Dunbrack's statistics |
| p_aa_pp (excluded after | Probability of amino acid at phipsi |
| cross-validation) | |
| ref | Amino acid reference energy for position |
| Additional amino | |
| acid level terms | |
| (structure derived, | |
| non-energetic) | Description |
| sasa | Solvent accessible surface area |
| hsasa | Hydrophobic solvent accessible surface |
| | area |

The dataset was completed by adding 1044 HLA-A2-incompatible peptides selected from IEDB training sets (i.e., those with reported affinities for HLA-A2>50,000 nM). Incorporating non-HLA-A2 binding peptides ensured that efforts addressed both TCR and MHC binding, as both directly contribute to immunogenicity and are dependent upon structure-determined energetic features. It is possible that accounting for both TCR and MHC binding together is necessary for predicting immunogenicity, as a peptide that binds weakly to an MHC protein could still prove immunogenic by possessing optimal features for TCR binding and vice versa. Moreover, peptide mutations can influence both TCR and MHC binding simultaneously as seen with differential T cell recognition of some “anchor fixed” shared tumor antigens.

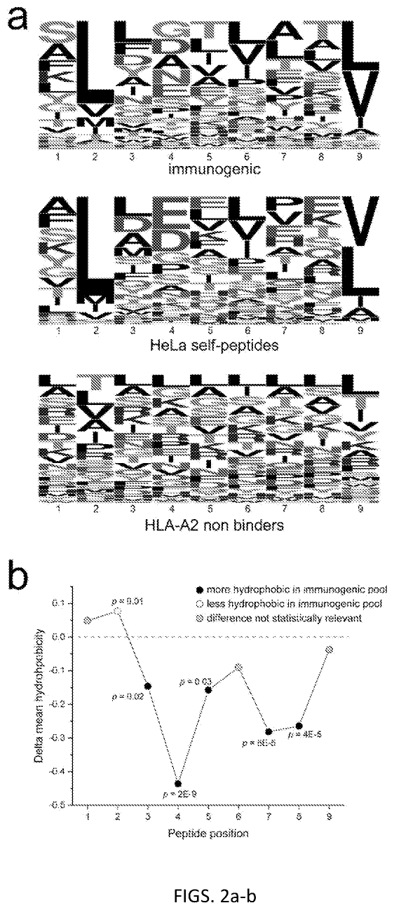

Amino acid distributions for the immunogenic, HeLa, and HLA-A2-incompatible peptides are shown in FIG. 2A. To ask if the dataset reflected previously noted distinctions between immunogenic and non-immunogenic peptides, the hydrophobicity of the peptides in the immunogenic and HeLa self-peptide pools was evaluated. Using the Wimley-White interface hydropathy index, the mean hydrophobicity for each peptide position in the two pools was determined. Comparing the results for the two showed that certain positions across the peptides were statistically more likely to be more hydrophobic in the immunogenic than the HeLa self-peptide pool, with the most pronounced differences at positions 4, 7, and 8 (FIG. 2B). These results, including the distinctiveness of p4, p7, and p8, support the conclusion that the peptide pools appropriately encompass both immunogenic and non-immunogenic peptides.

### Example 4

### A Neural Network to Predict Epitope Immunogenicity

Using the described structural modeling procedure and the database of peptides, an artificial neural network was constructed to predict the immunogenicity of nonameric peptides bound to HLA-A2, relying on structural and energetic features determined from three-dimensional models as the network inputs. Accordingly, structural models of all 3955 peptide/HLA-A2 complexes were generated. To describe the conformation-dependent physical properties of the peptides in the binding groove, the 18 terms in the Talaris2014 energy function commonly used for computational protein design were used to evaluate the energy of the entire peptide/HLA-A2 complex. The terms, listed in Table 1, account for features such as energies of attraction, repulsion, and solvation; energies of side chain and backbone hydrogen bonds; and energies and probabilities of side chain and backbone conformations. Nine terms from the same energy function for all nine positions in the peptide were also selected, selecting terms that emphasized atomic-level features and avoiding those descriptive of particular amino acids (e.g., tyrosine planarity). To the nine amino-acid level terms, total and hydrophobic solvent accessible surface areas were added. Overall then, 117 terms that describe each modeled peptide/HLA-A2 complex were used as network inputs. A binary classification system for each peptide in the dataset was used, classifying peptides identified from the IEDB as immunogenic and the HeLa and non-HLA-A2 binding peptides as non-immunogenic.

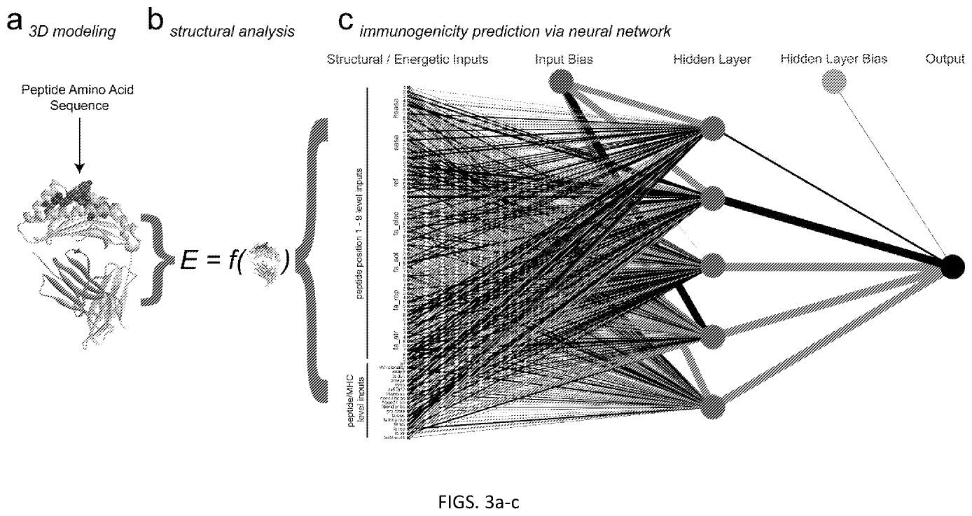

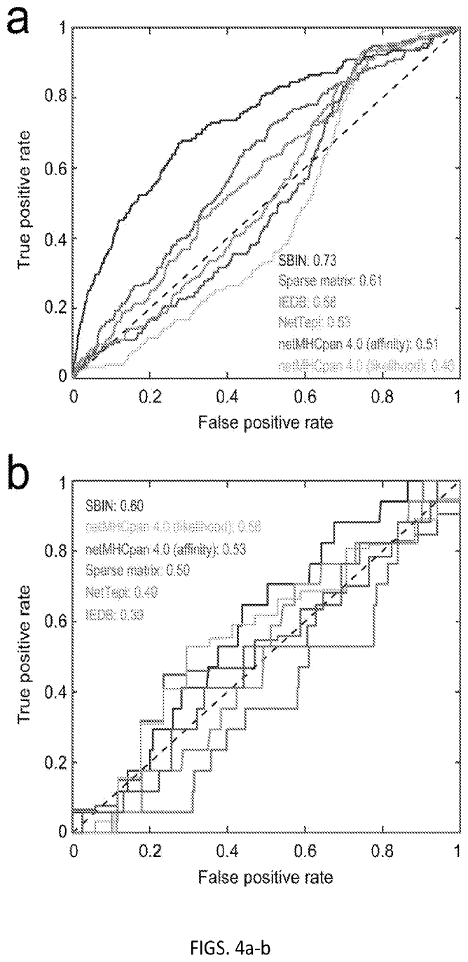

In developing the neural network, a nested 5-fold cross-validation procedure that eliminated redundant terms was used. The final model consisted of the 18 terms for the entire peptide/MHC complex and seven for each amino acid in the peptide, yielding 81 terms for network inputs, with five hidden neurons and two constant bias nodes (FIG. 3; Table 1). The average cross-validated area under the curve (AUC) in a receiver operating characteristic (ROC) plot was 0.69. The AUC values in the ROC plots give the probability that the neural network will more favorably score an immunogenic peptide compared to a non-immunogenic peptide. After training with the entire dataset, the final neural network (termed Structure Based Immunogenicity Network, or SBIN) classified all peptides used with a total AUC of 0.73 (FIG. 4A). SBIN outperformed a control network trained on the same 3955 peptides but encoded by a sparse matrix that considered only peptide sequence (AUC of 0.73 vs 0.61). For comparison to more established tools, the IEDB immunogenicity prediction tool classified the same 3955 epitopes with an AUC of 0.58, whereas NetTepi yielded a value of 0.54. netMHCpan 4.0, which emphasizes peptide binding as well as processing 18, performed more poorly, yielding AUC values of 0.51 for affinity and 0.46 for ligand likelihood.

### Example 5

### Significance of Network Inputs and Scores for Classifying Immunogenicity

Although interpreting the weights of inputs used within a neural network is difficult due to the complexity and nonlinear nature of the models, the weights of structural features used within the model can provide clues to their contributions in the evaluation of immunogenicity. For MHC binding, SBIN considered the impact of anchor residues 2 and 9 by assessing terms such as favorable van der Waals interactions at these positions to quantify if an epitope was compatible with HLA-A2. SBIN also focused on the interactions surrounding peptide position 3, likely considering peptide-MHC interactions in this constrained region of the HLA-A2 binding groove.

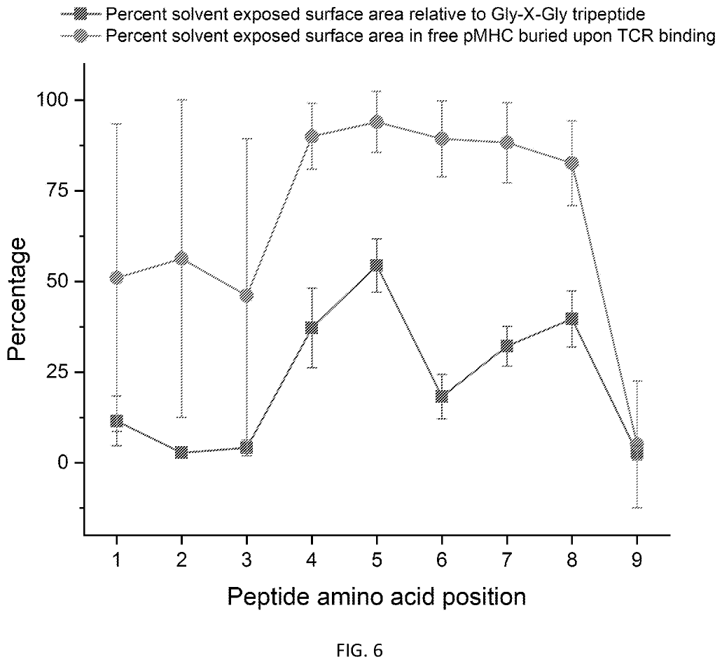

Consistent with the hypothesis that solvent exposed residues provide information regarding peptide immunogenicity by promoting TCR binding, SBIN emphasized hydrophobic SASA. Notably, the weights for hydrophobic SASA and hydrophobic solvation energy values at positions 5, 7, and 8 were in the top 10% of all weights in the neural network. These positions are typically considered ‘TCR facing’ in HLA-A2-presented nonameric peptides. Indeed, in the structural models used for training, positions 5, 7, and 8 had high degrees of solvent exposure, and crystallographic structures of TCRs bound to nonameric peptide/HLA-A2 complexes show that these positions on average bury more than 80% of their exposed surface upon receptor binding (FIG. 6).

One notable result from the analysis is that, excluding the non-HLA-A2 binding peptides, the total computed energies of the immunogenic complexes (as determined by the Talaris2014 total energy score used in the structural modeling) were higher than the non-immunogenic complexes (p<0.05). Although the difference is small (average of −560 Rosetta energy units for immunogenic complexes vs. −562 for non-immunogenic complexes), the energy reflects the entire peptide/MHC complex, of which the peptide is only approximately 2% by mass. This is believed to be an indicator of how structure and energy can influence the immunogenicity of neoantigens: amino acid substitutions that impart a higher energy onto a peptide/MHC (for example, by removing exposed charges and/or increasing exposed hydrophobic surface area) yield ligands that have more energy to release upon TCR binding, translating into stronger binding affinities.

### Example 6

Testing Performance on an Unrelated Neoantigen Data Set not Used in Training

SBIN outperformed IEDB, NetTepi, and netMHCpan 4.0 when classifying the training data. To further evaluate performance, 291 recently determined HLA-A2-restricted nonameric cancer neoantigens not used in training were inspected. These epitopes comprise a series of peptides that a variety of studies have examined in detail. Although only a subset of these have been reported as immunogenic in in vitro assays, the peptides nonetheless comprise a real-world test of the disclosed model. Although in general performance for all models was weaker with this dataset, SBIN again performed the strongest, with an AUC of 0.60, indicating a 60% likelihood of scoring an immunogenic peptide higher than a non-immunogenic peptide (FIG. 4B). SBIN substantially outperformed NetTepi (AUC 0.49) and the IEDB tool (AUC 0.39). netMHCpan performed better than IEDB and NetTepi, but still underperformed compared to SBIN, with AUCs of 0.56 for ligand likelihood and 0.53 for binding affinity. The sparse matrix model trained on sequence yielded an AUC of only 0.50. Although SBIN's edge compared to the other models against this dataset is relatively small, the fact that a relatively simple modeling strategy trained on limited datasets can outperform more highly developed platforms is notable. Additionally, the 291 neoantigens in this dataset are not equally well-characterized, and thus the upper limit of performance with this test set is unknown.

### Example 7

### Structural Evaluation of Select Immunogenic Neoantigens

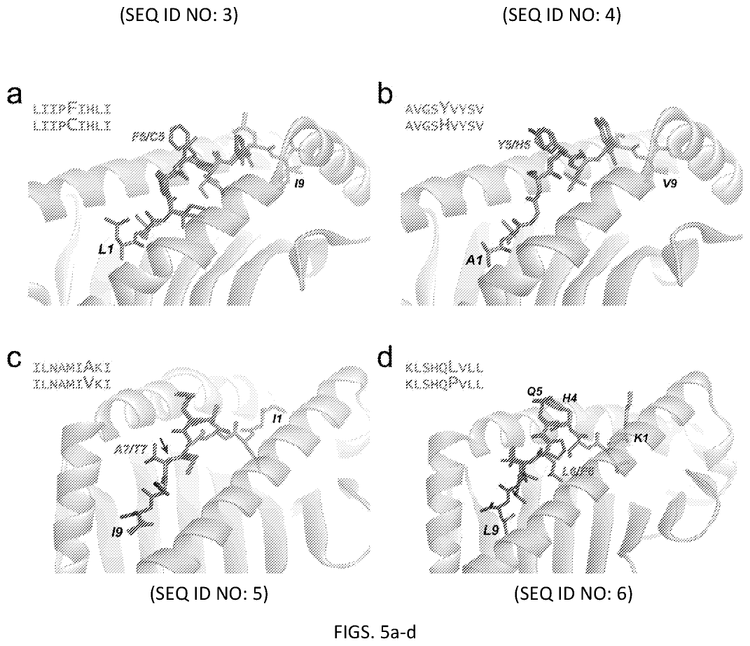

To examine how structural information can help inform the determination of immunogenicity, structural models of select immunogenic neoantigens and their wild-type counterparts were examined, focusing on immunologically well-characterized epitopes where mutations were not in primary anchor positions. The LIIPFIHLI (SEQ ID NO: 3) and AVGSYVYSV (SEQ ID NO: 4) epitopes were identified in melanoma patients to study heterologous T cell recognition of neoantigens, and SBIN predicted both neoantigen mutations would improve the immunogenicity of the wild-type peptide. Both neoepitopes harbor mutations at position 5, with LIIPFIHLI (SEQ ID NO: 3) replacing a cysteine with phenylalanine and AVGSYVYSV (SEQ ID NO: 4) replacing a histidine with a tyrosine. For both pairs of neoantigen and wild-type complexes, the structural models show the position 5 side chain to be almost fully exposed (FIG. 5A, 5B). The C→F mutation in LIIPFIHLI (SEQ ID NO: 3) results in the exposure of 90 Å² additional hydrophobic surface area, which could promote stronger TCR binding due to the hydrophobic effect. The H→Y mutation in AVGSYVYSV (SEQ ID NO: 4) results in a smaller increase in exposed hydrophobic surface (11 Å²). However, the mutation also removes an exposed positive charge whose burial would require overcoming an unfavorable desolvation penalty, while still providing opportunities for hydrogen bonding.

ILNAMIAKI (SEQ ID NO: 5) was identified in a study to identify immunogenic melanoma neoantigens and substitutes an alanine for a threonine at position 7. SBIN again predicted the neoantigen would have stronger immunogenicity compared to wild-type. The structural modeling suggests that the mutation simply removes the threonine side chain beyond the β carbon, with a small reduction in exposed hydrophobic surface area (−7 Å²). One structural difference that could have driven the prediction is the “unmasking” of a potential hydrogen bonding site on the peptide in response to the mutation, as the amide nitrogen of position 7 is predicted to be fully exposed in the neoantigen, yet sterically occluded in the wild-type peptide (FIG. 5C).

The neoantigen KLSHQLVLL (SEQ ID NO: 6) was identified in the same study as LIIPFIHLI (SEQ ID NO: 3) and AVGSYVYSV (SEQ ID NO: 4) and incorporates a proline to leucine at position 6 of the peptide. Although SBIN predicted the neoantigen mutation would improve immunogenicity relative to the wild-type epitope, both were ultimately assigned a low probability of immunogenicity. Position 6 side chains in nonamers presented by class I MHC proteins often point down towards the base of the peptide binding groove, where they can act as secondary anchors. This is predicted by the structural model for KLSHQLVLL (SEQ ID NO: 6) (FIG. 5D). The models again predict a small reduction in exposed hydrophobic surface area with the mutation (−16 Å²), but also the exposure of a new hydrogen bonding site at the p6 amide nitrogen. Despite this, SBIN may have considered the fully exposed histidine and glutamine at positions 4 and 5 as detrimental to immunogenicity.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method for predicting immunogenicity of a candidate peptide, the method comprising:
a. Obtaining a three-dimensional candidate structural representation of the candidate peptide bound to an antigen presenting molecule;
b. Obtaining a plurality of candidate measurements, wherein each candidate measurement is associated with at least one feature of the candidate structural representation; and
c. Predicting, with an electronic processor, the immunogenicity of the candidate peptide, wherein the electronic processor is configured to predict the immunogenicity of the candidate peptide based upon the plurality of candidate measurements.

2. The method of claim 1, wherein the electronic processor is further configured to predict the immunogenicity of the candidate peptide based upon a plurality of reference measurements,
wherein each reference measurement is associated with at least one feature of one or more reference structural representations,
wherein each reference structural representation is a three-dimensional representation of a reference peptide bound to the antigen presenting molecule,
wherein each reference peptide is a known immunogenic peptide or a known non-immunogenic peptide.

3. The method of claim 2, wherein the electronic processor is configured to predict the immunogenicity of the candidate peptide using a machine-learned model trained to predict immunogenicity of the candidate peptide using the plurality of reference measurements.

4. The method of claim 2, wherein the electronic processor is further configured to predict the immunogenicity of the candidate peptide based upon whether each reference peptide is an immunogenic peptide or a non-immunogenic peptide.

5. The method of claim 1, wherein the antigen presenting molecule is a class I MHC molecule or a class II MHC molecule.

6. The method of claim 5, wherein the antigen presenting molecule is HLA-A2.

7. The method of claim 1, wherein the plurality of candidate measurements and/or the plurality of reference measurements are selected from the group consisting of solvent accessible surface areas, solvation energies, hydrophobicity, electrostatic interactions, and van der Waals interactions.

8. The method of claim 1, wherein the candidate peptide is a neoantigen, a viral peptide, a non-mutated self peptide, or a post-translationally modified peptide.

9. A method for producing a vaccine, the method comprising:
a. Obtaining a plurality of candidate structural representations, wherein each of the candidate structural representations is a three-dimensional representation of a candidate peptide bound to an antigen presenting molecule;
b. Obtaining a plurality of candidate measurements for each candidate structural representation, wherein each candidate measurement is associated with at least one feature of each candidate structural representation;
c. Predicting, with an electronic processor, the immunogenicity of each candidate peptide based upon the plurality of candidate measurements for each candidate structural representation;
d. Producing a vaccine comprising one or more candidate peptides predicted to be immunogenic by the electronic processor.

10. The method of claim 9, wherein the electronic processor is further configured to predict the immunogenicity of each candidate peptide based upon a plurality of reference measurements,
wherein each reference measurement is associated with at least one feature of one or more reference structural representations,
wherein each reference structural representation is a three-dimensional representation of a reference peptide bound to the antigen presenting molecule,
wherein each reference peptide is a known immunogenic peptide or a known non-immunogenic peptide.

11. The method of claim 9, wherein the electronic processor is configured to predict the immunogenicity of each candidate peptide using a machine-learned model trained to predict immunogenicity of each candidate peptide using the plurality of reference measurements.

12. The method of claim 10, wherein the electronic processor is further configured to predict the immunogenicity of the candidate peptide based upon whether each reference peptide is an immunogenic peptide or a non-immunogenic peptide.

13. The method of claim 10, wherein the antigen presenting molecule is a class I MHC molecule or a class II MHC molecule.

14. The method of claim 13, wherein the antigen presenting molecule is HLA-A2.

15. The method of claim 10, wherein the plurality of candidate measurements and/or the plurality of reference measurements are selected from the group consisting of solvent accessible surface areas, solvation energies, hydrophobicity, electrostatic interactions, and van der Waals interactions.

16. The method of claim 10, wherein each candidate peptide is a neoantigen or a viral peptide.


SEQUENCE LISTING (66 sequences)

SEQ ID NO: 1 - protein, 9 aa, Artificial Sequence
  Synthetic
NLVPAVATV

SEQ ID NO: 2 - protein, 9 aa, Artificial Sequence
  Synthetic
LAGIGILTV

SEQ ID NO: 3 - protein, 9 aa, Artificial Sequence
  Synthetic
LIIPFIHLI

SEQ ID NO: 4 - protein, 9 aa, Artificial Sequence
  Synthetic
AVGSYVYSV

SEQ ID NO: 5 - protein, 9 aa, Artificial Sequence
  Synthetic
ILNAMIAKI

SEQ ID NO: 6 - protein, 9 aa, Artificial Sequence
  Synthetic
KLSHQLVLL

SEQ ID NO: 7 - protein, 9 aa, Artificial Sequence
  Synthetic
AAGIGILTV

SEQ ID NO: 8 - protein, 9 aa, Artificial Sequence
  Synthetic
ALGIGILTV

SEQ ID NO: 9 - protein, 9 aa, Artificial Sequence
  Synthetic
ALWGFFPVL

SEQ ID NO: 10 - protein, 9 aa, Artificial Sequence
  Synthetic
ALWGFVPVL

SEQ ID NO: 11 - protein, 9 aa, Artificial Sequence
  Synthetic
ALWGVFPVL

SEQ ID NO: 12 - protein, 9 aa, Artificial Sequence
  Synthetic
CINGMCWTV

SEQ ID NO: 13 - protein, 9 aa, Artificial Sequence
  Synthetic
CINGVCWTV

SEQ ID NO: 14 - protein, 9 aa, Artificial Sequence
  Synthetic
CINGVVWTV

SEQ ID NO: 15 - protein, 9 aa, Artificial Sequence
  Synthetic
CISGVCWTV

SEQ ID NO: 16 - protein, 10 aa, Artificial Sequence
  Synthetic
EAAGIGILTV

SEQ ID NO: 17 - protein, 10 aa, Artificial Sequence
  Synthetic
ELAGIGILTV

SEQ ID NO: 18 - protein, 10 aa, Artificial Sequence
  Synthetic
ALAGIGILTV

SEQ ID NO: 19 - protein, 10 aa, Artificial Sequence
  Synthetic
ELAGWGILTV

SEQ ID NO: 20 - protein, 9 aa, Artificial Sequence
  Synthetic
GILEFVFTL

SEQ ID NO: 21 - protein, 9 aa, Artificial Sequence
  Synthetic
GILGFVFTL

SEQ ID NO: 22 - protein, 9 aa, Artificial Sequence
  Synthetic
GILGLVFTL

SEQ ID NO: 23 - protein, 9 aa, Artificial Sequence
  Synthetic
GIWGFVFTL

SEQ ID NO: 24 - protein, 9 aa, Artificial Sequence
  Synthetic
GLCPLVAML

SEQ ID NO: 25 - protein, 9 aa, Artificial Sequence
  Synthetic
GLCTLVAML

SEQ ID NO: 26 - protein, 9 aa, Artificial Sequence
  Synthetic
ILKEPVHGV

SEQ ID NO: 27 - protein, 9 aa, Artificial Sequence
  Synthetic
FLKEPVHGV

SEQ ID NO: 28 - protein, 9 aa, Artificial Sequence
  Synthetic
YLKEPVHGV

SEQ ID NO: 29 - protein, 9 aa, Artificial Sequence
  Synthetic
ILSALVGIL

SEQ ID NO: 30 - protein, 9 aa, Artificial Sequence
  Synthetic
ILSALVGIV

SEQ ID NO: 31 - protein, 9 aa, Artificial Sequence
  Synthetic
IMDQVPFSV

SEQ ID NO: 32 - protein, 9 aa, Artificial Sequence
  Synthetic
ITDQVPFSV

SEQ ID NO: 33 - protein, 9 aa, Artificial Sequence
  Synthetic
KVAEIVHFL

SEQ ID NO: 34 - protein, 9 aa, Artificial Sequence
  Synthetic
KVAELVHFL

SEQ ID NO: 35 - protein, 9 aa, Artificial Sequence
  Synthetic
KVAELVWFL

SEQ ID NO: 36 - protein, 9 aa, Artificial Sequence
  Synthetic
LGYGFVNYI

SEQ ID NO: 37 - protein, 9 aa, Artificial Sequence
  Synthetic
LLYGFVNYI

SEQ ID NO: 38 - protein, 9 aa, Artificial Sequence
  Synthetic
LLYGFVNYV

SEQ ID NO: 39 - protein, 9 aa, Artificial Sequence
  Synthetic
LLFGKPVYV

SEQ ID NO: 40 - protein, 9 aa, Artificial Sequence
  Synthetic
LLFGYPVYV

SEQ ID NO: 41 - protein, 9 aa, Artificial Sequence
  Synthetic
NLVPMCATV

SEQ ID NO: 42 - protein, 9 aa, Artificial Sequence
  Synthetic
NLVPMGATV

SEQ ID NO: 43 - protein, 9 aa, Artificial Sequence
  Synthetic
NLVPMVAAV

SEQ ID NO: 44 - protein, 9 aa, Artificial Sequence
  Synthetic
NLVPMVATV

SEQ ID NO: 45 - protein, 9 aa, Artificial Sequence
  Synthetic
NLVPMVAVV

SEQ ID NO: 46 - protein, 9 aa, Artificial Sequence
  Synthetic
NLVPMVHTV

SEQ ID NO: 47 - protein, 9 aa, Artificial Sequence
  Synthetic
NLVPQVATV

SEQ ID NO: 48 - protein, 9 aa, Artificial Sequence
  Synthetic
NLVPSVATV

SEQ ID NO: 49 - protein, 9 aa, Artificial Sequence
  Synthetic
NLVPTVATV

SEQ ID NO: 50 - protein, 9 aa, Artificial Sequence
  Synthetic
NLVPVVATV

SEQ ID NO: 51 - protein, 10 aa, Artificial Sequence
  Synthetic
RLYQNPTTYI

SEQ ID NO: 52 - protein, 10 aa, Artificial Sequence
  Synthetic
KLYQNPTTYI

SEQ ID NO: 53 - protein, 9 aa, Artificial Sequence
  Synthetic
SLANTVATL

SEQ ID NO: 54 - protein, 9 aa, Artificial Sequence
  SLFNTVATL
SLFNTVATL

SEQ ID NO: 55 - protein, 9 aa, Artificial Sequence
  Synthetic
SLLMWITQA

SEQ ID NO: 56 - protein, 9 aa, Artificial Sequence
  Synthetic
SLLMWITQC

SEQ ID NO: 57 - protein, 9 aa, Artificial Sequence
  Synthetic
SLLMWITQL

SEQ ID NO: 58 - protein, 9 aa, Artificial Sequence
  Synthetic
SLLMWITQS

SEQ ID NO: 59 - protein, 9 aa, Artificial Sequence
  Synthetic
SLYLTVATL

SEQ ID NO: 60 - protein, 9 aa, Artificial Sequence
  Synthetic
SLYNTIATL

SEQ ID NO: 61 - protein, 9 aa, Artificial Sequence
  Synthetic
SLYNTVATL

SEQ ID NO: 62 - protein, 9 aa, Artificial Sequence
  Synthetic
SLYNVVATL

SEQ ID NO: 63 - protein, 9 aa, Artificial Sequence
  Synthetic
VLHDDLLEA

SEQ ID NO: 64 - protein, 9 aa, Artificial Sequence
  Synthetic
VLRDDLLEA

SEQ ID NO: 65 - protein, 9 aa, Artificial Sequence
  Synthetic
YMFPNAPYL

SEQ ID NO: 66 - protein, 9 aa, Artificial Sequence
  Synthetic
RMFPNAPYL